(19) United States
(12) Patent Application Publication
(10) Pub. No.: US 2024/0110147 A1
(43) Pub. Date: Apr. 4, 2024

(54) METHOD AND DEVICE FOR DELIVERING ELECTROMAGNETIC ENERGY (LIGHT) IN A FLUID COMPRISING AN ORGANISM

(71) Applicant: FORO VENTURES GMBH, Fällanden (CH)

(72) Inventor: Jean-Louis Roux Dit Buisson, Fällanden (CH)

(21) Appl. No.: 18/554,002

(22) PCT Filed: Apr. 5, 2022
(86) PCT No.: PCT/EP2022/059027

§ 371 Date: Oct. 5, 2023

(30) Foreign Application Priority Data
Apr. 6, 2021 (CH) 00354/21

(51) Int. Cl. C12M 1/00

(52) U.S. Cl. CPC C12M 45/07; C12M 31/10

(57) ABSTRACT

Devices and methods for influencing an organism included in a fluid by a light source immersed in the fluid, wherein a motion of the light source in the fluid is or can be pre-set and/or adjusted. Culturing and killing/debilitating are examples of influencing an organism. A light source immersed in a culture milieu provides fouling free supply of photonic energy to the culture milieu growing phototropic or mixotrophic organisms and operating in continuous or semi continuous or batch or semi-batch mode. The light source for delivering light energy in a facility for culturing an organism, a device for delivering light energy in a facility for culturing an organism, a facility 30 culturing an organism, and a method for operating a facility for culturing an organism are provided.

### BACKGROUND OF THE INVENTION

### Field of the Invention

The present invention relates to devices, in particular a light source and devices including the light source, for delivering electromagnetic energy in a facility for influencing, in particular culturing but also killing or debilitating, an organism comprised in a fluid, in particular a liquid, in a volume in open air or contained environment with the electromagnetic energy. The present invention relates further to a facility for influencing, in particular culturing but also killing or debilitating, an organism comprised in a fluid, in particular a liquid, in a volume in open air or contained environment with electromagnetic energy, and to a method of operating a facility for influencing, in particular culturing but also killing or debilitating, an organism comprised in a fluid, in particular a liquid, in a volume in open air or contained environment with electromagnetic energy.

In particular, the invention relates to devices and methods for controlling, in particular pre-setting and/or adjusting, a motion of a light source in a fluid, in particular a liquid.

Important applications of the invention are culturing organisms in a fluid, in particular a liquid, and purifying fluids, in particular liquid such as water.

### Description of Related Art

The productivity of facilities including an organism in a fluid and a lighting system that is at least partly immersed in the fluid is unsatisfactory in many cases. Downtime due to maintenance, in particular for maintaining lighting systems at least partly immersed in the fluid, and/or due to providing electromagnetic energy in a manner that is suboptimal in terms of the position of the light source within the fluid, of emitted wavelength and/or of changing condition within the fluid are examples of reasons that reduce the productivity of a facility for influencing, in particular culturing but also killing or debilitating, an organism comprised in a fluid and having a lighting systems that is at least partly immersed in the fluid.

This is in particular the case in facilities for culturing organisms, such as biomass, especially microalgae biomass, farmed in a volume in open air or contained environment, such as a photobioreactor (PBR), The cultured organisms are usually photosynthesizing organisms. in particular phototropic and/or mixotrophic organisms, especially photosynthesizing algal biomass.

Fouling, in particular fouling on elements of the facility used for delivering or transmitting photonic energy to the organisms, is an important issue in facilities for culturing organisms and a reason for increased downtime and decreased productivity of such facilities.

A worldwide concern for increasing global levels of carbon dioxide (CO₂) in the atmosphere has emerged in the last ten years. Countries and governments are continuously trying to implement regulatory frameworks in order to incite efforts for reducing the overall emissions of CO₂ and/or equivalent greenhouse gases. Biological carbon sequestration though photosynthesis is a natural way to recycle carbon that has been recently extensively explored for addressing this problem.

Further, worldwide fossil fuel deposit depletion has pushed researching for alternatives to products that are currently processed from fossils. In certain applications where high amounts of fuels are needed remotely from sources of supply (e.g., forward military bases or remote exploratory camps experience), costs associated with conventional fuels are high, primarily due to expenses involved in fuel delivery and associated pollution due to transportation means. Therefore, alternatives to produce fuels at the point of use, rather than transporting them to the desired site have been investigated to reduce those costs. In this effort, biofuels such as biodiesel have been identified as a possible alternative for replacing fossil fuel consumption without increasing the CO₂ content of the atmosphere. However, the process involved in creating biofuel from biomass is currently expensive relative to the process of extracting and refining petroleum.

A number of strategies are focused on methods to increase carbon dioxide uptake in biological systems such as green plants through sunlight and CO₂ uptake while research went on for optimizing production yields, diversifying and valorising the biomass by-products resulting from photosynthesis. However, the industrial development of those strategies has been hampered by many difficulties in transposing those experimental methods into scalable and/or cost effective solutions. In particular, control of the main parameters affecting the rate of photosynthesis, e.g., a favourable temperature, intensity and wavelength of light, and availability of nutrients such as carbon dioxide has proven to be delicate for closed system applications (e.g., photobioreactors), whereas open ponds to grow biomass suffer from risks of contamination and exhibit high operating costs, for example.

Among phototrophic microorganisms, microalgae is one of the most efficient organisms for converting solar energy using carbon dioxide as growth nutrient and is an efficient producer of oxygen and biomass. Valuable components such as carbohydrates, sugars, proteins and fat can be harvested from the biomass and converted into high value added products such as protein, fine chemicals or energy supplies such as methane or biodiesel and other fuels used in thermal cycle engines for generating movement, in transportation, essentially.

It is known that microalgae productivity in production facilities is limited by four major factors: availability of light, availability of nutrients, temperature, and downtime of the production facility. The need for maintenance, in particular maintenance due to fouling of surfaces through which light is transmitted to the organisms for the photosynthetic process, is one of the main reasons for relevant downtime. Historically, most efforts have been invested in developing the optimum nutrients for any specific microalgae, notably by saturating the photosynthesizing system with CO₂. Land-based (e.g., ponds) microalgae culture plants, while showing some effectiveness in capturing CO₂, are limited by available land space, water supplies (mainly due to evaporation), external contamination (e.g., other species, bird dejections), productivity (not operable at night) and costs associated with the processing of huge quantities of water. Optimal temperature conditions for efficient biomass production are usually selected in accordance with the climatic conditions prevailing in a chosen site. Yet, even in such sites, winter and night temperatures, as well as morning hour temperatures pose serious limitations to growth rates.

Further, UV exposure of the microalgal culture in outdoors production plants results in the oxidation of the microalgae at the surface of the water. Attempts to solve these problems led to the creation of shallow ponds or raceways. However, such shallow water approaches engender high evaporation and saline deposits, which also reduce the efficacy of continuous outdoor growth. Overall, weather, diurnal cycles, invasion by opportunistic species and external pollutions further aggravate the difficulties of mass microalgae culturing in outdoor settings.

Photobioreactors (PBRs) for photosynthesizing biomass culture provide a compact infrastructure designed to address the above problems. The scale-up of photobioreactors to achieve a commercially viable production of algae products is hampered by the limitation of available lighting, both in terms of light delivery, distribution, energy expenditure, and—again— of fouling of the surfaces through which light travels to the milieu, such as reactors walls, walls of inserted equipment to distribute light, such as glass or plastic tubes inside which light dispensing devices are inserted, or light sources directly in contact with the growth culture. For instance, current methods of mass cultivation of micro-algae include translucent fiberglass cylinders, polyethylene bags, carboys and tanks under artificial lighting and/or natural illumination in greenhouses. During the microalgae growing process the organisms multiply and the culture density increases, and light ends up not being able to penetrate below a few centimetres of depth below the surface of the algae culture thereby decreasing the volumetric productivity of the system.

Various PBRs use lighting systems that are arranged inside the PBR to improve illumination of the biomass. However, little effort has been made so far to address the associated issue of increased fouling, in particular due to fouling on the lighting system.

U. S. 2009/0029445 discloses a biological growth reactor including a mixer, a mixing chamber and a reaction chamber including a light distributing and fluid dispensing rod. U.S. 2009/0291485 discloses a culture system including a culture tank, a rotatable light array and a rotational drive. WO 2011/154886 A1 discloses an internal light delivery system in a culture tank operated continuously or semi continuously. US 2020/0146220 A1 discloses a self-powered energy output system in a waterproof casing. The waterproof casing is configured to be neutrally buoyant in an enclosure including one or more photosynthetic cultures and the energy output system is configured to harvest energy from water movement. US 2021/0054420 A1 discloses processes for the production and processing of polyhydroxyalkanoates (PHA) from carbon sources and a liquid including microorganism culture including PHA-containing biomass. The processes may include a step of suspending a light-emitting device in the liquid. The light-emitting device may be free-floating and it is powered from the liquid itself by inducing a voltage in the liquid. WO 2020/046206 A1 and JP 2012-115236 A disclose the use of phosphorescent material for illuminating a liquid including biomass, wherein the phosphorescent material is immersed in the liquid. In WO 2020/046206 A1, the phosphorescent material is arranged on a scaffold that is immersed in the liquid. In JP 2012-115236, the phosphorescent material is arranged on a base material. Phosphorescent material and base material form a phosphorescent bead, wherein the base material has a thermal expansion rate that allows the phosphorescent bead to float or sink in dependence of a temperature change. WO 2010/0279395 A1 discloses various artificial light systems for containers for cultivating microorganisms therein. The artificial light systems disclosed are arranged in a centre tube that is mounted to the container. The artificial light system and the centre tube extend from a top of the container to a bottom of the container through the whole container. KR 20160037503 A discloses a similar artificial light system.

To our knowledge, none of state-of-the-art PBRs having a lighting system that is at least partly arranged in the liquid including the biomass addresses the issue of fouling.

The lightning system is subject to fouling due to the shear stress caused by the flow of biomass, for example algae, over fixed or moving parts, more precisely over parts that move relative to the biomass. This causes frequent and costly downtime for maintenance, and a loss of productivity as the light sources can be covered by proteins released by dead micro-organisms, or physical accumulation (adherence) of organisms on the light source, which is detrimental to operating costs and efficiency.

Further, light reduction within the PBR decreases biomass productivity and, therefore, the photosynthetic efficiency of the cultivation system and the economics of the production process as maintenance and downtime are required more frequently. Additionally, PBR biofouling leads to a series of further undesirable events including changes in cell pigmentation, culture degradation, and contamination by invasive microorganisms; all of which can result in the cultivation process having to be stopped.

Designing PBR surfaces with proper materials, functional groups or surface coatings to prevent biomass, such as microalgal, adhesion are possible approaches for addressing the biofouling issue. However, such approaches are costly and it may be that the materials, functional groups and surface coatings need to be biomass and/or liquid specific, in particular if the biomass needs to be “bio” certified.

### SUMMARY OF THE INVENTION

It is an object of the invention to provide devices and methods to overcome drawbacks of state-of-the-art devices, in particular drawbacks that result in a reduced productivity of facilities for influencing an organism comprised in a fluid with a lighting system that is at least partly immersed in said fluid.

In particular, it is an object of the invention to provide a light source and a device for delivering electromagnetic energy in a fluid, wherein a light emitting portion, in particular the light source, is immersed in the fluid and wherein the light source and the device allow to determine and/or adjust a behaviour of the light emitting portion in the fluid.

According to an important aspect of the invention, it is the motion of the light emitting portion in the fluid that is adjusted. However, operational settings of the light emitting portion in the fluid may be adjusted in addition or alternatively, for example.

It is a further object of the invention to provide a related facility including a light emitting portion, in particular the light source, immersed in a fluid and a method of operating such a facility.

In particular, it is an object of the invention to provide a method of operating a facility for influencing, organisms, a facility for influencing organisms and devices for delivering electromagnetic (photonic) energy (also called light dispersing device, lighting system etc.) in such a facility.

An important field of application of the invention is in the field of culturing organisms comprised in a fluid. If the invention is applied in this field of application, the organism is a photosynthesizing organism and the organism is influenced for culturing (farming, growing, reproducing). In particular if the invention is applied in this field of application, it is an object of the invention to provide a method of operating a facility for culturing photosynthesizing organisms, a facility for culturing photosynthesizing organisms and devices for delivering electromagnetic in such a facility that partially or totally eliminate known fouling causes and hence reduce fouling in the facility, in particular fouling due to a device for delivering electromagnetic energy that is immersed at least partly in a fluid, usually liquid, including the photosynthesizing organisms. The fluid including the photosynthesizing organisms is also called culture milieu.

If the application is applied in the field of culturing, the organism that is a photosynthesizing organism is usually is biomass, especially microalgae biomass, farmed in a volume in open air or contained environment of the facility.

Independent of the field of application of the invention, the facility can be of a contained volume type, of a tubular reactor-type, or of any other type including a volume containing organisms.

The facility may be operated in continuous or semi-continuous flow or batch or semi-batch mode.

If the application is applied in the field of culturing, the facility may be a photobioreactor (PBR), for example the contained volume type PBR as described in WO 2011/154886 A1 and WO 2020/046206 A1, a tubular reactor-type such as those supplied by Schott Glass and operated in Roquette' Freres's Klotze unit, or of any other type including a volume containing photosynthesizing organisms.

At least one of the objects of the invention is achieved by the claimed method, facility and device for delivering light energy.

Although the concepts of the invention are disclosed in the following with respect to culturing photosynthesizing organisms, said concepts may be used in any facility including an organism in a fluid, in particular liquid, and having a purpose that is different from the purpose of culturing, such as purifying.

In simple terms, devices and methods according to the invention achieve at least one of the above objects by determining and/or adjusting the relative motion of the part or parts of the lighting system immersed in the fluid, in particular the light source immersed in the fluid, wherein the determination/adjustment of the relative motion does not only include the relative velocity between the fluid and the parts of the lighting system immersed in the fluid, but also the direction from which and the manner the fluid flows over said parts, for example.

In simple terms, the motion of the parts depends on the forces acting on the parts, in particular the weight force, the buoyancy force, the drag force and the force exerted by the flow on the overflowed parts. Therefore, the motion of the parts depends in particular on their weight and shape (including volume and surface properties). Further, the motion of the parts may be influenced by means external to the part or parts immersed, such as a magnetic field or means for pulling the parts.

The invention makes use of the above for providing devices and methods that are configured for determining and/or adjusting the motion of the part or parts immersed in the liquid in a manner that is beneficial for the operation of the facility. In particular, at least one of the above-mentioned forces is determined or adjusted by at least one of a specifically designed part or parts, in particular with respect to weight and/or shape, by providing means for adjusting the part or parts, in particular the weight and/or shape, and by providing means external to the part or parts that influence a resulting force acting on the part or parts in the fluid during operation of the facility.

According to another aspect of the invention, the part or parts are equipped for delivering the desired electromagnetic energy in the fluid in a reliable manner. In principle, this aspect of the invention may be realised in devices and methods according to the invention that do not include the above-summarized aspect concerning the determined and/or adjusted motion of the part or parts. However, in many embodiments the part or parts are equipped for delivering the desired electromagnetic energy in a reliable manner and without interfering the determined and/or adjusted motion of the part or parts.

A further aspect of the invention concerns the maintenance of the part or parts immersed in the fluid and/or adjustment of their operational parameters. In principle, this aspect of the invention may be realised in devices and methods according to the invention that do not include the above-summarized aspect concerning the determined and/or adjusted motion of the part or parts. However, in many embodiments of the devices and methods this aspect and the aspect concerning the determined and/or adjusted motion of the part or parts are realized. In addition, the aspect concerning the delivering of the desired electromagnetic energy in a reliable manner may be realized in embodiments.

A first aspect of the invention concerns a method for operating a facility configured for influencing, in particular culturing (farming, growing, reproducing), an organism with the aid of electromagnetic energy.

The facility operated by the method includes an outer wall and a volume in which the organism is influenced with electromagnetic energy, for example a volume for culturing the organism and in which electromagnetic energy is provided. In the following, the invention is described with respect to its application in the field of culturing an organism. However, this does not rule out its use in methods, facilities and devices for influencing an organism in a manner that does not or not primarily contribute to culturing.

The method includes a step of providing a fluid in the volume, wherein the fluid includes the organism (“culture milieu”).

The organism is usually a photosynthesizing organism, in particular a phototropic and/or mixotrophic organisms. The organism can be considered as biomass. In embodiments, the organism is photosynthesizing algal biomass, in particular photosynthesizing microalgae biomass. However, the concepts of the invention are also applicable to facilities including organisms different from photosynthesizing organism comprised in a fluid, in particular liquid, and/or to facilities that do not have the purpose of culturing an organism. A facility for purifying a liquid is an example of a facility in which and in which method of operating the concepts of the invention may be used as well.

The method includes further a step of providing a light source in the fluid, wherein the light source is configured to provide electromagnetic energy to the fluid. The electromagnetic energy is or contributes to the electromagnetic energy used to influence the organism.

“Providing a light source in the fluid” means that the light source is immersed in the fluid. This means a casing of the light source, more precisely a casing that is an integral part of the light source, is in direct contact with the fluid.

The light source provided includes at least one of a rechargeable internal energy supply and a wired connection to a power supply, in particular a primary power supply, that is outside of the light source.

The rechargeable internal energy supply is in particular an energy supply that is rechargeable in a wireless (cordless, non-wired) manner, for example by electromagnetic waves. In particular, the internal energy supply is rechargeable from an energy source that is arranged outside of light source, in particular outside of the volume.

The light source and the facility may be configured for the rechargeable internal energy supply to be recharged outside of the fluid, in particular outside of the fluid only.

The rechargeable internal energy supply may be a battery that is rechargeable in a wireless (cordless) manner, for example by inductive charging.

In embodiments in which the light source includes a wired connection to a power supply that is outside of the light source, the power supply may even be arranged outside of the volume.

The light source includes a light emitting unit in which power from the rechargeable internal energy supply or from the power supply that is outside of the light source is converted to the light energy provided by the light source. In many embodiments, the light source includes an LED.

The light source may include a plurality of (this means more than one) light emitting units. The light emitting units may differ in the light energy (wavelength) they emit. The light emitting unit may be controllable independently from each other. In particular, they may be switched on and off independently from each other.

The light source may be as disclosed according to any embodiment of the second aspect of the invention that relates to the facility and/or according to any embodiment of the third aspect of the invention that relates to a device for delivering light.

Independent of the concrete realization of the light source, the light source is provided in the fluid in a manner that it can move in the fluid and the method includes a step of determining a motion of the light source in at least one direction in the fluid.

In other words, the light source is not firmly installed in the volume when the facility is operated. Rather, the facility in general and the light source in particular allow for a movement of the light source with respect to the outer wall.

The motion of the light source in the fluid may be determined for having a desired residence time inside the fluid, for example. This is in particular the case if the light source travels in the fluid, for example from a light source feeding unit to a light source extracting unit or from a storage volume to a collection volume.

The motion of the light source in the at least one direction is determined by at least one of:
A step of adjusting the light source.
In particular, the physical, for example the hydrostatic and/or hydrodynamic, properties of the light source are adjusted.
A step of selecting a light source from a plurality of light sources that differ in their motion in the fluid.
A step of providing a guiding element and applying a pulling force to the light source via the guiding element.
A step of applying a magnetic field in the volume, wherein the light source includes a magnet.

In other words, the facility, or rather the light source or the device for delivering electromagnetic energy as the case may be, is designed for a resulting force acting on the light source in the liquid leading to a set motion of the light source, said set motion being determined in the step of determining a motion of the light source.

The motion of the light source is in particular set to a defined relative velocity between the light source and the fluid. The relative velocity may be set to zero or to a velocity that is slow enough for the flow of the fluid over the light source being a laminar flow. In other words, the relative velocity of the light source may be set, in dependence of the shape of the light source and the orientation of the light source in the relative flow field, for the light source in the fluid being below the laminar-turbulent transition.

For example, the Reynolds number may be considered in the step of determining a motion of the light source. In particular, the Reynolds number may be small, in particular below the laminar-turbulent transition range.

The Reynolds number may vary during operation, for example when the concentration of the organisms changes due to operation (semi-batch and batch modes, continuous mode while the milieu grows to its operating concentration of micro-organism).

In an embodiment, the step of adjusting the light source, if present, is usually carried out prior to provide the light source in the fluid.

In an embodiment, the step of selecting the light source, if present, is usually carried out prior to provide the light source in the fluid.

In an embodiment, the method includes at least one of:
The step of adjusting the light source, wherein the step of adjusting the light source is a step of adjusting the light source to a characteristic of the fluid.
The step of selecting a light source, wherein the step of selecting the light source includes a selection in dependence of a characteristic of the fluid.
The step of applying a pulling force to the light source, wherein the pulling force applied depends on a characteristic of the fluid.
The step of applying a magnetic field, wherein a force generated on the light source by the magnetic field applied depends on a characteristic of the fluid.

The temperature of the fluid, the density of the fluid, the flow field in the volume, the flow velocity in the volume, and the Reynolds number are examples of characteristics of the fluid.

In an embodiment, a resulting force acting on the light source in the fluid is determined in the step of determining the motion of the light source.

The method may include further a step of providing a light source that is designed in a manner that the determined resulting force acts on the light source when immersed in the fluid and during operation of the facility.

The resulting force is determined for the light source carrying out the determined motion in the fluid.

In particular, the resulting force is determined to set a velocity differential between the light source and the fluid.

The velocity differential may be a vertical velocity differential.

Independent of the concrete realization of the method, at least one of the weight force and the buoyancy force acting on the light source in the fluid is determined in the step of determining the motion of the light source, in an embodiment. In particular, the light source may be adjusted or selected for having a determined weight. For example, this may be done by the use of a weight element as described below and/or by a shape of the light source having a volume as described below.

The step of determining a motion of the light source in the fluid may be carried out under consideration of the operation mode of the facility.

In embodiments, the facility is operated, at least temporarily, in one of the following modes:
The fluid is at rest or includes a flow field with no significant flow in vertical direction and the light source floats in or with the fluid. In this operation mode, the light source may be adjusted or selected for the weight force and buoyancy force acting on the light source to be essentially equal in strength (and opposite in direction).
A flow field with no significant flow in vertical direction may result from a flow field for mixing the constituents of the fluid, namely the organism and the nutrients, and/or it may result from a fluid inflow and a fluid outflow, wherein the fluid may be the fluid with or without the organism or a nutrition fluid, for example.
The flow field may be caused by a step of generating a directed flow of the fluid in the portion of the volume.
The fluid is at rest or includes a flow field with no significant flow in vertical direction and the light source sinks or rises slowly in the fluid. In this operation mode, the light source may be adjusted or selected for the weight force, buoyancy force and the drag resistance acting on the light source being approximately equal in strength, only.
The fluid includes a flow field with a relevant vertical component and one of the following applies:
   The light source floats in the fluid. In this operation mode, the light source may be adjusted or selected for the weight force and buoyancy force acting on the light source being essentially equal in strength (and opposite in direction).
   The light source moves vertically in the same direction as the fluid but with an absolute velocity that is smaller than the absolute velocity of the fluid in vertical direction. In this operation mode, the light source may be adjusted or selected for the weight force, the buoyancy force and the drag resistance summing up to a force having a direction opposite to the vertical flow direction of the fluid and having a strength that is smaller than the strength of the vertical component of the force exerted on the light source by the fluid flowing over the light source, this means the force exerted by the flow on the overflowed light source.
   Preferably, the resulting relative velocity between the light source and the fluid is such that there is, at least predominantly, laminar flow over the light source.
   In particular in this operation mode, the light source may include a shape that favors laminar flow of the fluid over the light source.
   The absolute velocity may be considered as the velocity relative to the outer wall, for example.
   The light source moves vertically in the direction opposite to the vertical flow direction of the fluid. In this operation mode, the light source may be adjusted or selected for the weight force, the buoyancy force and the drag resistance summing up to a force having a direction opposite to the vertical flow direction of the fluid and having a strength that is larger than the strength of the vertical component of the force exerted on the light source by the fluid flowing over the light source, this means the force exerted by the flow on the overflowed light source.
   Preferably, the resulting relative velocity between the light source and the fluid is such that there is, at least predominantly, laminar flow over the light source.
   In particular in this operation mode, the light source may include a shape that favors laminar flow of the fluid over the light source.
A flow field with a relevant vertical component may result from a fluid inflow and a fluid outflow, wherein the fluid may be the fluid with or without the organism or a nutrition fluid, for example.
A flow field with no significant flow in vertical direction may result from a flow field for mixing the constituents of the fluid, namely the organism and the nutrients, and/or it may result from a fluid inflow and a fluid outflow, wherein the fluid may be the fluid with or without the organism or a nutrition fluid, for example.
The flow field may be caused by a step of generating a directed flow of the fluid in the portion of the volume.

Independent of the operation mode, the light source may be configured to move freely in the fluid, to move freely in the at least one direction in the fluid, or to move in a guided manner in the at least one direction in the fluid.

A light source can be configured to move freely in one direction by the use of a guiding element, for example. Such an embodiment is disclosed below in detail. A light source that swims freely in the fluid in all directions is an embodiment of a light source that moves freely in the fluid.

A light source that is connected to and moved by a guiding element is an example of a light source that is configured to move in a guided manner. Also this embodiment is disclosed below in detail.

Laminar flow of the fluid over the light source is advantageous in terms of avoiding fouling on the light source. As pointed out above, setting the relative velocity between the light source and the fluid is one approach for generating laminar flow over the light source. Choosing a shape of the light source that favours laminar flow of the fluid over the light sources is another approach. Therefore, the step of determining a motion of the light source in the fluid according to any embodiment of this step may include to provide a light source that has a shape that favours laminar flow of the fluid over the light source, in embodiments. In particular, the light source may be adjusted to or selected for having a shape that favours laminar flow. A shape that favours laminar flow is in particular beneficial in operation modes in which the light source would have a relative velocity with respect to the fluid that would cause turbulent flow if the shape of the light source were not a shape that favours laminar flow.

In other words, the shape of the light source may be determined in consideration of the relative velocity between the light source and the fluid to be below the laminar-turbulent transition.

A shape favours laminar flow over the body having said shape if the shape does not present any non-smooth obstacle to the fluid flowing around the shape.

For example, the shape may be ovoid-shaped, drop-shaped or spherical.

In an embodiment, the method includes at least one of a step of feeding a light source into the volume in an automated manner and a step of extracting a light source out of the volume in an automated manner.

In an embodiment including the step of feeding a light source into the volume, it is at least one of an adjusted and selected light source that is fed into the volume.

In an embodiment including the step of extracting a light source out of the volume and the step of feeding a light source into the volume, a light source of the volume may be replaced by a light source that is more appropriate for the conditions in the volume. For example, the extracted light source was adjusted or selected in a first step of determining a motion and the fed light source was adjusted or selected in a second step of determining, wherein a change of a characteristic of the fluid, said change has taken place between the first and second step of determining a motion of the light source, is considered in the second step of determining a motion of the light source.

The step of feeding may be carried out in a manner that does not need access of a user of the facility to the light source.

The step of extracting may be carried out in a manner that does not need access of a user of the facility to the light source.

For example, the facility may be designed that there is no direct access or no access at all to the volume during operation of the facility. The latter is the case if the facility defines a closed system during operation, for example. In particular in facilities designed in this manner, this means with no direct access or no access at all to the volume during operation, the step of feeding and or the step of extracting may contribute to avoid fouling on the light source.

In particular by the step of extracting the light source out of the volume, the time exposure of the light source in the fluid may be limited. In addition, the light delivered to the fluid may be held constant by feeding a clean light source in the volume.

As will be disclosed in detail below, the facility may provide a recirculating system for light sources. The recirculating system may be equipped with a surface cleaning unit and allowing hence for long lasting fouling free run times, especially in continuous flow operations, without decrease in productivity per unit time and volume.

In embodiments including a step of cleaning the light source, the light source fed into the volume may be a light source that was extracted in the step of extracting a light source out of the volume and that was cleaned in the step of cleaning the light source.

Optionally, a decision whether a light source is extracted or not may depend on at least one of the kind of the light source, a characteristic of the light source, and the operational settings of the light source.

Optionally, a decision whether a light source is fed into the volume or not may depend on at least one of the kind of the light source, a characteristic of the light source, and the operational settings of the light source. At least one of the kind of the light source, a characteristic of the light source, and the operational settings of the light source may be changed before feeding the light source into the volume.

Independent of the concrete realization of the embodiment in which the method includes the step of adjusting the light source, the light source may be adjusted by attaching an adjustment portion to the light source or by replacing an adjustment portion of the light source.

Similarly, and independent of the concrete realization of the embodiment in which the method includes the step of selecting a light source, the selected light source may differ from the non-selected light sources of the plurality of light sources in an adjustment portion.

In an embodiment, the weight of the light source is adjusted, in particular by replacing the adjustment portion, and/or a light source of a specific weight is selected, for example by the light source including a specific adjustment portion.

Independent of the embodiment of the method, a plurality of light sources is provided usually.

In embodiments, the light sources of said plurality of light sources are separate light sources. For example, this is the case if the light sources swim freely in the fluid, in particular liquid.

In other words, a light source of the plurality of light sources is not connected to any other light source of the plurality of light sources. This means also that the light sources move in the medium in an independent manner, except possible collisions, screening effects, etc.

However, there are also embodiments in which a light source of the plurality of light sources is connected with at least another light sources of the plurality of the light source. For example, this is the case in embodiments including the guiding element if more than one light source is connected to the guiding element.

Light sources of the plurality of light sources may also form an open or closed light chain.

At least two of the light sources of a light chain may differ in the wavelength they emit. For example, at least one light source may emit in the blue wavelength range and at least another light source may emit in the red wavelength range. In addition, at least a further wavelength range may emit in the amber or orange wavelength range.

For example, the light chain may form an alternating sequence of two or more light sources that emit at different wavelength.

For example, the light chain may be a cluster of at least two light sources that emit at different wavelength.

The plurality of light sources may be a plurality of identical light sources.

However, in embodiments, a plurality of light sources of a first kind and plurality of light sources of a second kind are provided, wherein a light source of the first kind distinguishes from a light source of the second kind in its physical properties, wherein the physical properties in which the light source of the first kind distinguishes from the light source of the second kind determines the condition of the fluid and/or the condition in the fluid for which the light source is optimized.

In particular, a light source of the first kind distinguishes from a light source of the second kind in at least one of:
The wavelength range emitted by the light source.
The weight of the light source,
A shape of the light source, in particular a shape of the light source that influences at least one of the flow of the fluid over the light source, the drag force generated by the flow of the fluid over the light source (more generally, the ability of the light source to be transported be the fluid in motion), the buoyancy of the light source in the fluid.
A strength of a magnet of the light source.

Alternatively or in addition, a light source of the first kind may distinguishes from a light source of the second kind in any feature of the light source disclosed with respect to the step of determining a motion of the light source.

The plurality of light sources from which a light source is selected in the step of selecting a light source from a plurality of light sources may include a light source of the first kind and a light source of the second kind.

In an embodiment, independent of the manner the step of determining a motion of the light source is determined and independent of the concrete embodiment of the light source or plurality of light sources, the method includes a step of maintenance of the light source during operation of the facility.

The step of maintenance of the light source during operation of the facility includes usually at least one of:
The step of feeding the light source into the volume.
In an embodiment, the light source may be fed into the volume by the facility including an inlet into the volume, in particular an inlet connected to a tubing, wherein the inlet and, as the case may be, the tubing is/are configured for the light source to pass.
This may be the case in particular in embodiments in which the method includes at least one step of maintenance of the light source that is carried out outside the volume and outside an outer wall enclosing the volume. Examples of steps of maintenance of the light source that may be carried out outside of the volume are the steps of charging, cleaning, assorting and identifying, for example.
In an embodiment, the outer wall of the facility may enclose the volume for culturing the organism and a storage volume for the light source. In this embodiment, the step of feeding may include feeding a light source from the storage volume into the volume.
The storage volume may be arranged relative to the volume such that a light source stored in the storage volume may enter the volume due to gravity after an element holding the light source in the volume, for example a closure or fastener, has been activated or manipulated.
For example, the storage volume may be arranged vertically above the volume.
As mentioned, the step of feeding, independent of the concrete realization of the feeding into the volume, may be used to feed the light source adjusted in the step of adjusting the light source and/or the light source selected in the step of selecting a light source from a plurality of light sources into the volume. Therefore, the step of feeding may not only allow for feeding a clean light source into the volume but also for feeding a light source into the volume that is different from a light source already present in the volume. In particular, the light sources differ as disclosed with respect to the light source of the first kind and the light source of the second kind.
If the step of maintenance of the light source includes further the step of extracting a light source out of the volume, the step of maintenance of may lead to a replacement of a light source by another, optionally different, light source.
The step of extracting the light source out of the volume.
In an embodiment, the light source may be extracted out of the volume by the facility including an outlet from the volume, in particular an outlet connected to a tubing, wherein the outlet and, as the case may be, the tubing is/are configured for the light source to pass.
This may be the case in particular in embodiments in which the method includes at least one step of maintenance of the light source that is carried out outside the volume and outside an outer wall enclosing the volume. Examples of steps of maintenance of the light source that may be carried out outside of the volume are the steps of charging, cleaning, assorting and identifying, for example.
In an embodiment, the outer wall of the facility may enclose the volume for culturing the organism and a collection volume for the light source. In this embodiment, the step of extracting may include extracting a light source from the volume into the collection volume.
The collection volume may be an integral part of the volume.
The collection volume may be arranged relative to the volume such that a light source may enter the collection volume due to gravity.
For example, the collection volume may be arranged vertically below the volume.
A step of charging the light source, wherein the light source is charged outside of the volume.
The step of charging may be carried out in an automated manner. In particular, it may be carried out in a manner that does not need access of a user of the facility to the light source.
In an embodiment, the step of charging is carried out in a charging station of the facility that is arranged outside of the volume and in connection to the volume via a tubing.
The charging station may be arranged outside of an outer wall surrounding the volume. However, the charging station may be an integral part of the facility. In particular, the charging station may be an integral part of the closed system defined during operation by the facility if the facility is of a kind that defines such a closed system.
The charging may be a wireless (cordless) charging.
The charging may be carried out via a contact between a conductive contact of the light source and a conductive contact of the charging station. The contact between the conductive contact of the light source and the conductive contact of the charging station is a direct contact between said contacts, in an embodiment. This means that there is no wire or other element arranged between said contacts during charging.
A step of cleaning the light source, wherein the light source is cleaned outside of the volume.
The step of cleaning the light source may be carried out in an automated manner. In particular, it may be carried out in a manner that does not need access of a user of the facility to the light source.
In an embodiment, the step of cleaning is carried out in a cleaning unit of the facility that is arranged outside of the volume and in connection to the volume via a tubing. The cleaning unit may be arranged outside of an outer wall surrounding the volume. However, the cleaning unit may be an integral part of the facility. In particular, the cleaning unit may be an integral part of the closed system defined during operation by the facility if the facility is of a kind that defines such a closed system.
The cleaning causes a removal of adherences and warrants an optimal surface for diffusion of light in the fluid.
A step of assorting the light sources of the first and second kinds in embodiments of the method including the step of providing a plurality of light sources of a first kind a plurality of a second kind.
The step of assorting the light sources may be carried out in an automated manner. In particular, it may be carried out in a manner that does not need access of a user of the facility to the light source.
In an embodiment, the step of assorting is carried out in an assorting unit of the facility that is arranged outside of the volume and in connection to the volume via a tubing.
The assorting unit may be arranged outside of an outer wall surrounding the volume. However, the assorting unit may be an integral part of the facility. In particular, the assorting unit may be an integral part of the closed system defined during operation by the facility if the facility is of a kind that defines such a closed system.
The step of assorting is carried out after a step of identifying, for example, as described in the following, usually.
The step of assorting may sort out a light source with malfunction.
A step of identifying the light source.
The step of identifying the light source may be carried out in an automated manner. In particular, it may be carried out in a manner that does not need access of a user of the facility to the light source.
For example, the step of identifying may include an electronic or optical reading-out of an identification unit of the light source or it may determine a property of the light emitted by the light source.
In an embodiment, the step of identifying is carried out in an identification station of the facility that is arranged outside of the volume and in connection to the volume via a tubing.
The identification station may be arranged outside of an outer wall surrounding the volume. However, the identification station may be an integral part of the facility. In particular, the identification station may be an integral part of the closed system defined during operation by the facility if the facility is of a kind that defines such a closed system.
In embodiments, the step of identifying is applied if the plurality of light sources of a first kind and the plurality of light sources of the second kind are provided, this means light sources of at least two kinds that distinguish as pointed out above for example, are provided. In these embodiments, the step of identifying may be a step of determining whether a light source is a light source of the first kind or a light source of the second kind.
The step of identifying may identify a light source with malfunction.
A step of adjusting an operational setting of the light source.
The step of adjusting an operational setting of the light source may be carried out in an automated manner. In particular, it may be carried out in a manner that does not need access of a user of the facility to the light source.
In an embodiment, the step of adjusting an operational setting of the light source is carried out in a programming station of the facility that is arranged outside of the volume and in connection to the volume via a tubing.
The programming station may be arranged outside of an outer wall surrounding the volume. However, the programming station may be an integral part of the facility. In particular, the programming station may be an integral part of the closed system defined during operation by the facility if the facility is of a kind that defines such a closed system.
The step of adjusting an operational setting of the light source may be carried out by the light source including a controller configured to control and adjust the operational setting of the light source remotely. In other words, a characteristic of the light emitted by the light source can be adjusted remotely.
In particular, the programming station and the controller may be configured for programming or re-programming the controller. Programming and re-programming can be carried out wirelessly or through connection. However, re-programming at least is usually carried out without direct access to the light source and its controller. In particular, re-programming at least may be carried out without extracting the light source or its controller from the closed system that the facility may define during operation.
Alternatively or in addition, the step of adjusting an operational setting of the light source may include any adjustment disclosed with respect to the step of affecting the light source to adjust the driving force and/or the step of selecting a light source from a plurality of light sources.

In an embodiment, the light source includes a magnet configured for positioning the light source in at least one of the above-listed steps, this means in at least one of the feeding unit, the extracting unit, the charging station, the cleaning unit, the assorting unit, the identification station, and the programming station.

In other words, the method may include a step of positioning the light source in at least one of the feeding unit, the extracting unit, the charging station, the cleaning unit, the assorting unit, the identification station, and the programming station by use of a magnet.

In particular, the magnet may be used (configured) for positioning the light source during at least one of charging, cleaning, and programming or re-programming.

A second aspect of the invention concerns a facility for influencing, in particular culturing (farming, growing, reproducing), an organism with electromagnetic energy. In the following, the invention is described with respect to its application in the field of culturing an organism. However, this does not rule out its use in methods, facilities and devices for influencing an organism in a manner that does not or not primarily contribute to culturing.

In embodiments, the facility is a photobioreactor (PBR).

The facility may include any component in any embodiment disclosed with respect to the method and/or components that are configured to carry out any step in any embodiment disclosed with respect to the method.

The method may include providing the facility and/or a component of the facility in any embodiment disclosed in the following and/or any step corresponding to functionalities of the facility and/or any component of the facility in any embodiment disclosed in the following.

Any suitable photosynthesizing microorganism may be cultured in the facility with the device for delivering light energy according to the third aspect as source of light energy and/or the method of operating according the first aspect. For example, the facility is suitable to grow aqueous micro organisms, in particular a photosynthesizing bio-culture, in particular microalgae growing in salty or non-salty water (e.g. Viridaeplantae (Chlorella, chlorophycophyta), Chrysophycophyta (golden algae), Rodophyta (red algae), stramenopiles (diatoms and algae, from the Bacillariophyceae family, phaecophytophyta brown algae), 24hotosynthetic prokaryotes such as cynobacteria, photosynthesizing eukaryotes excluding charales family, Spirulina, Nanochloropsis, Prorocentrum minimum), at a large scale in a continuous, semi continuous flow or batch mode.

Examples of further photosynthesizing microorganisms that can be advantageously grown in a facility using the invention are vegetable tissues and monocellular organisms containing chloroplasts, photosynthesizing bacteria and algae such as those described in Gudin et al., 1986, “Bioconversion of solar energy into organic chemicals by microalgae” in Advances in Biotechnological processes 6, pp 73-110.

In an embodiment, a facility, in particular a PBR, using the invention is used for the production of photo-autotrophic cells, examples of which include Chlorella, Scenedemus, Chlamydononas, Cyanobacteria, and Spirulina.

In another embodiment, cells cultured in a facility, in particular a photobioreactor, using the invention include those which have had their genome modified by genetic engineering techniques in order to produce specific metabolites, or to improve CO₂ fixation, or to improve other performance parameters.

The choice of the operating conditions of the device will depend on the photosynthesizing cell culture and the operating objectives such as yield, nature of the metabolites and polysaccharides excreted from the microorganisms (Metting et al., 1986, Enzyme Microbiol. Technol., 8, pp 386-394).

The facility includes an outer wall, a volume for culturing (influencing) the organism with electromagnetic energy, a fluid arranged in said volume, wherein the fluid includes the organism, and a device for delivering electromagnetic energy (also called a lighting system, a light dispersing device etc.) to the fluid. The device for delivering light includes a light source that is arranged (immersed) in the fluid, wherein the light source includes at least one of a rechargeable internal energy supply and a wired connection to a power supply, in particular a primary power supply, that is outside of the light source or even of the volume. The light source can move in the fluid in at least one direction, wherein a motion of the light source in the at least one direction in the fluid is determined by at least one of:
The light source being a light source adjusted to a characteristic of the fluid.
The light source or—more general—the device for delivering electromagnetic energy may be adjusted to a characteristic of the fluid and/or to an operation mode of the facility as disclosed with respect to the method, the device for delivering electromagnetic energy itself and/or the light source itself (third aspect).
The light source being a light source selected from a plurality of light sources in dependence of a characteristic of the fluid.
The plurality of light sources and the selection of the light source in dependence of a characteristic of the fluid may, that may dependent on an operation mode of the facility as disclosed with respect to the method, may be as disclosed with respect to the method, the device for delivering electromagnetic energy itself and/or the light source itself (third aspect).
The facility including a guiding element, wherein the light source and the guiding element are configured for applying a pulling force to the light source via the guiding element.
The guiding element and the corresponding light source may be as disclosed with respect to the method, the device for delivering electromagnetic energy itself and/or the light source itself (third aspect).
A magnetic field applied in the volume, wherein the light source includes a magnet. The magnetic field and the magnet be as disclosed with respect to the method, the device for delivering electromagnetic energy itself and/or the light source itself (third aspect).

The motion of the light source may be determined as disclosed in any embodiment of the method and/or the third aspect.

The light source or the device for delivering electromagnetic energy may be realized according to any embodiment disclosed with respect to the method and/or the third aspect.

In embodiments, the fluid is a liquid.

The rechargeable internal energy supply and related features and/or components of the light source and/or of the facility may be or configured as disclosed with respect to the method.

The wired connection to a power supply and the power supply may be as disclosed with respect to the method and/or to the third aspect.

The meaning of “arranged in the fluid” and “the light source can move in the fluid in at least one direction in at least a portion of the volume” is the same as disclosed with respect to the method.

In an embodiment, the motion of the light source is determined by a resulting force acting in the fluid on the light source, this means on the adjusted, selected, pulled and/or exposed to the magnetic field light source.

The resulting force and the related light source can be determined as disclosed with respect to the method.

In other words, the facility includes a light source that is configured for having a determined, this means set, desired, pre-defined etc., resulting force acting on it when being placed in the fluid and/or the facility and the light source are configured for the light source having a determined, this means set, desired, pre-defined etc., resulting force acting on it when being placed in the fluid and operating the facility. The former is in particular the case in embodiments including the adjusted and/or selected light source. The latter is in particular the case in embodiments including the guiding element and/or the magnetic field.

Independent of the concrete realization of the facility, the motion of the light source in the at least one direction in the fluid that is determined (in short “the determined motion”) includes a determined relative velocity between the light source and the fluid, in an embodiment.

The absolute value of the determined relative velocity and its direction may be as disclosed in any embodiment with respect to the method. The direction of the relative velocity may be given by the orientation of the light source in the relative flow field.

The determined motion of the light source may include a determined velocity differential between the light source and the fluid, in particular a determined vertical velocity differential.

Independent of the concrete realization of the facility, the motion of the light source is determined by the light source being adjusted and/or selected for at least one of a given weight force and a given buoyancy force acting on the light source in the fluid.

In particular, the light source may be adjusted and/or selected for having the weight force and/or the buoyancy force as disclosed with respect to the method in any embodiment and/or the third aspect.

In particular, the light source may be adjusted and/or selected and, as the case may be, the facility may be configured for the facility being operable in any of the operation modes disclosed with respect to the method.

In an embodiment, the facility includes at least one of a light source feeding unit configured to feed the light source into the volume in an automated manner and a light source extracting unit configured to extract the light source from the volume in an automated manner.

The light source feeding unit and the light source extracting unit may be as disclosed with respect to the method and/or they may be configured to carry out any step of the method related to these units.

In particular, the light source feeding unit may be configured in a manner that the light source can be fed into the volume during operation of the facility and without the need for access of a user of the facility to the light source.

In addition or alternatively, the light source feeding unit may be configured to feed the light source into the volume without manipulating the outer wall.

In an embodiment, the light source feeding unit is configured to feed light sources that differ in the wavelength range emitted according to the proportion of wavelengths needed for culturing the organism.

In particular, the light source extracting unit may be configured in a manner that the light source can be extracted from the volume during operation of the facility and without the need for access of a user of the facility to the light source.

In addition or alternatively, the light source extracting unit may be configured to extract the light source from the volume without manipulating the outer wall.

The device for delivering light may consist of the light source.

The device for delivering light, or its light source at least, may be separate from any other component of the facility except the fluid in which it is immersed. This is in particular true when the light source is arranged in the volume for delivering light to the fluid.

The facility may include a plurality of light sources as disclosed with respect to the method and/or the third aspect.

In particular the facility may include a plurality of light sources of the first kind and a plurality of light sources of the second kind as disclosed with respect to the method.

The facility may include a plurality of devices for delivering light in the number of the number of the plurality of light sources.

However, the facility may include one or more devices for delivering light that may include more than one light source. For example, this may be the case in the embodiment in which the device for delivering light includes one or more guiding elements if more than one light source is engaged to the guiding element or to some/all of the guiding elements.

In an embodiment, the device for delivering light consists of a plurality of light sources that are separate from any other component of the photobioreactor, except from the fluid in which they are immersed.

In embodiments, the facility includes at least one of the following components, wherein the components are as disclosed in the description of the method and/or configured to carry out any step of the method related to the corresponding component:
A flow generating system configured to generate at least in a portion of the volume a directed flow of the fluid.
If there is a directed flow in the said portion at least, for example generated by the flow generating system, the device for delivering light may be configured for the light source to be moved along the flowing direction of the directed flow.
The charging station.
If the facility includes a charging station for the device for delivering light, the device, in particular the light source, includes a charging unit via which the rechargeable internal energy supply may be charged.
The charging unit may be as disclosed with respect to the third aspect. In particular, the charging unit (and the charging station) are configured for wireless charging. In an embodiment, the charging unit includes a conductive contact and the charging station includes a conductive contact, wherein the conductive contacts are arranged in a manner that they can come into contact. In particular, the conductive contact of the charging unit is arranged at an outer surface of the light source and the conductive contact of the charging station is arranged at a surface that is exposed towards a region in which the light source may stay or through which the light source my travel.
The cleaning unit.
The identification station.
The identification station is configured to determine at least one property of the light source. Therefore, the device for delivering light, in particular the light source, may include an electronic or optical identification unit as disclosed with respect to the third aspect, for example. The identification station may then be configured to determine the at least one property by reading-out the identification unit. Alternatively, the identification station may be configured to determine directly a property of the light source, for example a characteristic of the light emitted by the light source, such as the wavelength or intensity emitted, the weight of the light source, a shape or dimension of the light source or a magnetic property of the light source. In this case, the device may not need the identification unit.
The assorting unit.
The assorting unit may be configured to assort light sources according to at least one property identified by the identification station.
A pipe system for transporting the light source from the volume to a component that handles the light source and that is arranged outside of an outer wall surrounding the volume, and/or from a component to another component, and/or from a component to the volume.
In embodiments, the pipe system is a circulating system.
In particular, the pipe system may be configured to connect components that handle the light source and that are arranged outside of an outer wall surrounding the volume in a manner that the light source can travel from the volume to a first component, from the first component to a second component (if present), from the second component (if present) to a third component (if present) and so on and finally back to the volume.
The programming station.

In an embodiment, the facility includes the light source feeding unit in any embodiment disclosed, the light source extracting unit in any embodiment disclosed and the charging station in any embodiment disclosed, wherein the light source feeding unit, the light source extracting unit and the charging station are an integral part of the facility and wherein the facility is configured, for example by including a pipe system that may be a circulating system, to deliver the light source from the light source extracting unit to the charging station and from the charging station to the light source feeding unit.

The embodiment may include further the cleaning unit in any embodiment disclosed, wherein the cleaning unit is an integral part of the facility and wherein the facility is configured to deliver the light source, for example by the mentioned pipe system, from the light source extracting unit to the cleaning unit and from the cleaning unit to the charging station.

In addition, the embodiment may include further the identification station in any embodiment disclosed, a plurality of light sources in any embodiment disclosed, and the assorting unit in any embodiment disclosed, wherein the identification station and the assorting unit are an integral part of the photobioreactor and wherein the facility is configured to deliver, for example by the mentioned pipe system, the plurality of light sources from cleaning unit to the identification station, from the identification station to the assorting unit and, while maintaining the sorting, from the assorting unit to the light source feed unit or, in dependence of the position of the charging station, to the charging station and then to the light source feed unit.

In an embodiment, the facility includes a plurality of light sources including in any embodiment disclosed and including the charging unit, the light source feeding unit in any embodiment disclosed, the light source extracting unit in any embodiment disclosed, the charging station in any embodiment disclosed, the cleaning unit in any embodiment disclosed, the identification station in any embodiment disclosed, and the assorting unit in any embodiment disclosed, wherein the facility is configured, for example by including a pipe system that may be a circulating system, for the plurality of light sources to pass the following units and stations in a consecutive manner: the light source extracting unit, the cleaning unit, the identification station, the charging station, the assorting unit, and the light source feeding unit.

However, a different arrangement of the units and stations in a consecutive manner is conceivable. For example, the charging station may be arranged in front of the identification station and the assorting unit.

Independent of the concrete realization of the facility and its components, the light source, in an embodiment, includes a light emitting portion and an adjustment portion, in particular the light emitting portion and the adjustment portion in any embodiment disclosed with respect to the method (first aspect) and/or third aspect, wherein the motion of the light source in the at least one direction in the fluid is determined by the adjustment portion and/or depends on the adjustment portion.

The adjustment portion may be detachable from the light emitting portion.

A third aspect of the invention concerns a device for delivering electromagnetic energy (also called a light dispersing device, a lighting system etc.) in a facility for influencing, in particular culturing (farming, growing, reproducing), an organism with electromagnetic energy, in particular in a facility according to the second aspect. The device includes a light source. In the following, the invention is described with respect to its application in the field of culturing an organism. However, this does not rule out its use in methods, facilities and devices for influencing an organism in a manner that does not or not primarily contribute to culturing.

The light source may include a casing that is air- and water-tight for the device being suitable for delivering electromagnetic energy in a facility for influencing, in particular culturing, an organism.

Depending on the facility, the light source is configured to work under pressure and the casing may be water-tight at operational pressure of the facility. In particular, the casing may be water-thigh up to a pressure of 40 bars.

The casing is transparent in the wavelength (energy) range to be delivered to the organism.

Suitable materials that can be used to form the casing include but are not limited to transparent polymers with or without internal and/or external coatings. The external surface of the coated or non-coated casing may be biocompatible with the organisms in cultivation and ideally exhibits lipophobic properties in order to avoid agglomeration of matters, such as AMC18 ™ (Advanced Materials Components).

In particular, the casing may include or be made of a biocompatible polymer or copolymer having optionally ultraviolet resistance properties or a material having ultraviolet resistance properties. Polycarbonates, acrylic resins, polypropylene, polyethylene, polyvinylchloride and glass, and/or the same coated with specific coatings exhibiting light filtering and transparency are examples of suitable materials. However, other transparent materials and coatings can also be envisioned.

The casing may be rigid, semi-rigid, flexible.

The dimensions of the casing are any suitable dimensions and forms for the purpose of delivering light in the fluid (culture milieu) in any manner according to the invention.

The material of the casing must withstand temperatures up to 80 degrees Celsius without loss of water-tightness, physical integrity, transparency, or organoleptic surface properties, in particular anti-adhesion for proteins and biomass, sugars, lipids, and chemical compounds usually found in the milieu while cultivating phototropic or mixotrophic organisms.

The device for delivering light energy may include any feature in any embodiment disclosed in the description of the method with respect to device for delivering light energy or the light source and/or any feature that is configured to carry out any step in any embodiment disclosed in the description of the method with respect to device for delivering light energy or the light source.

The device for delivering light energy may include any feature in any embodiment disclosed in the description of the facility with respect to device for delivering light energy or the light source.

The method may include providing the device for delivering light energy and/or the light source in any embodiment disclosed in the following and/or any step corresponding to functionalities of the device and/or its light source in any embodiment disclosed in the following.

The facility may include the device for delivering light energy and/or the light source in any embodiment disclosed in the following and/or the facility may be configured to enable the device and/or the light source to fulfil any function disclosed in the following when being part of the facility.

Various solutions to determine and/or adjust the motion of the light source in the fluid for improving the productivity of the facility, in particular for addressing the fouling issue on a light source immersed in the fluid including the organism to be cultured, have been presented with respect to the method of operating a facility (first aspect) and with respect to the facility itself (second aspect).

In the device, these solutions may be implemented by the device including a light source, wherein the light source includes a rechargeable internal energy supply and/or the device includes a wired connection of the light source to a power supply, in particular a primary power supply, that is outside of the light source, wherein at least one of the following applies:
The light source includes a light emitting portion and an adjustment portion for adjusting a motion of the light source in a fluid, wherein the adjustment portion is attached or attachable to the light emitting portion.
In particular the light source may include a light emitting portion and an adjustment portion attachable to the light emitting portion, wherein the motion of the light source in the fluid is determined and/or can be adjusted by adjusting or replacing the adjustment portion.
The adjustment portion may be as disclosed according any embodiment with respect to the method, the facility and/or the light source itself.
The light emitting portion may be as disclosed according to any embodiment with respect to the method, the facility and/or the light source itself
The device includes a plurality of light sources that differ in their motion in a fluid. The plurality of light sources may be the plurality of light sources according to any embodiment disclosed with respect to the method, the facility and the light source itself. In particular, the device may include at least one light sources of the first kind and at least one light sources of the second kind as described with respect to the method and the facility.
The light source includes an engagement portion configured to engage the light source with a guiding element of the device, wherein the guiding element defines a direction of motion along which the engaged light source can move and the engagement portion is configured to move the engaged light source together with the guiding element along the direction or to allow a movement of the engaged light source along the guiding element in the direction.
The guiding element may be or include a guiding wire.
The engagement portion may be as disclosed with respect to the light source itself
The light source includes a magnet configured to interact with an external magnetic field in a manner that a motion of the light source in a fluid can be influenced. The magnet configured to interact with an external magnetic field may be the corresponding magnet in any embodiment disclosed with respect to the light source itself.
The light source includes a weight element.
The weight element may be the corresponding weight element in any embodiment disclosed with respect to the light source itself.

Independent of the concrete realization of the device, the device and/or the light source can be according to any embodiment disclosed with respect to the method, the facility and the light source itself.

The device for delivering light may consist of the light source in any embodiment disclosed or of a plurality of light sources in any embodiment disclosed. However, this is not the case in embodiments including the guiding element, for example.

In an embodiment, the device includes or consists of a plurality of light sources.

The plurality of light sources may be separate light sources or some of them may be connected, for example via the guiding element, or all of them may be connected, for example via the guiding element.

The plurality of light sources may be identical light sources.

Alternatively, the device for delivering light may include light sources of different kinds. In particular, the device may include one or more light source of a first kind and one or more light source of a second kind as described with respect to the method.

In the light source itself, at least some of the mentioned solutions to determine and/or adjust the motion of the light source in the fluid for improving the productivity of the facility, in particular for addressing the fouling issue on a light source immersed in the fluid including the organism to be cultured, may be present. In particular, this is the case if the light source includes a rechargeable internal energy supply or the light source does not include a rechargeable energy supply, for example the light source does not include an energy supply at all, and being configured to establish a wired connection to an external energy supply, and if a motion of the light source in a fluid is determined by the light source including at least one of:
A light emitting portion and an adjustment portion attachable or attached to the light emitting portion.
In particular the light source may include a light emitting portion and an adjustment portion attachable to the light emitting portion, wherein the motion of the light source in the fluid is determined and/or can be adjusted by adjusting or replacing the adjustment portion.
The adjustment portion may be as disclosed with respect to the method and/or the facility.
The light emitting portion may be as disclosed with respect to the method and/or the facility.
For the adjustment portion being attachable to the light emitting portion, the light emitting portion may have an attachment point (or attachment region) and the adjustment portion may have a corresponding attachment point (attachment region). Each attachment point may include magnetic properties that allows for an attachment of the adjustment portion to the light emitting portion, for example. Other attachment mechanisms are conceivable, for example an attachment mechanism including a bayonet lock, a thread or a snap lock.
An engagement portion configured to engage the light source with a guiding element, in particular a guiding element as disclosed with respect to the method, the facility and/or the device for delivering electromagnetic energy.
The guiding element may be or include a guiding wire.
The engagement portion may be configured to move the engaged light source together with the guiding element along the direction. This means the light source is firmly mounted to the guiding element and the device for delivering light energy may be equipped for moving the light source by pulling the guiding element.
Alternatively, the engagement portion may be configured to allow a movement of the engaged light source along the guiding element in the direction. This means that the guiding element guides the light source in at least one direction that is radial to the direction defined by the guiding element but that it is not possible to apply a force along the direction defined by the guiding element to the light source by the guiding element.
In this alternative embodiment, the light source is moved by the force resulting from the drag force, the gravity force, the buoyancy force, friction force and the optional magnetic force.
A magnet configured to interact with an external magnetic field.
In particular, the magnet may be configured to adjust the motion of the light source in the fluid, for example to fine-tune the motion, to adjust the resulting forces acting on the light source to a changed characteristic of the fluid or to bring the light source to a specific position, for example to an outlet of the volume, to one of the units and stations disclosed with respect to maintenance of the light source etc. This means that the magnet is different in strength and its arrangement in or at the light source from a magnet used for mount parts of the light source to each other or for mount the light source to another element of the device for delivering electromagnetic energy, for example. The magnet is also different from any magnet generated during operation of the light source, for example due to current, for example.
The magnet configured to interact with an external magnetic field may be designed for the light source taking a defined orientation in an essentially homogeneous magnet field applied in the volume.
The external magnetic field may be generated by magnets and/or coils arranged outside of the volume and the magnet of the light source may be adjusted to the external magnetic field generated in this manner.
The magnet configured to interact with an external magnetic field may be the magnet configured for positioning the light source in at least one of the feeding unit, the extracting unit, the charging station, the cleaning unit, the assorting unit, the identification station, and the programming station. However, in embodiments, the light source does not include the magnet configured to interact with an external magnetic field but includes the magnet configured for positioning the light source in at least one of the mentioned units and stations, only.
A weight element.
The weight element is configured to adapt the overall weight of the light source.
The weight element is usually an element of its own that has the only purpose to influence the overall weight of the light source. In other words, the weight element is different from elements of the light source that fulfill a specific purpose such as the light emitting portion, the rechargeable internal energy supply, a casing, a charging unit, a controller, an identification unit, an engagement portion, a magnet etc.
The weight element may be fixed inside the casing surrounding a light emitting unit, this means the unit of the light source in which the light is generated.
The weight element may be fixed inside the light emitting portion of the light source. Alternatively, the weight element may be part of the adjustment portion.
The weight element may be a weight to position the light source in the fluid, in particular if it is an upstream or downstream fluid, and/or to influence the up or down movement of the light source in the fluid.
For example, the weight element may be capable of vertically positioning the device at different heights in the fluid by playing on the weight element at a specific temperature of the fluid.
The weight element can include or consist of any solid, liquid or gel-like material.
The weight element may be amorphous.
The weight element may include a weight distribution. In other words, the weight element includes a weight distribution that is not homogeneous. For example, the density of the weight element is not constant in the weight element.
The opposite of a weight element is a volume filled with a material having a lower density than the fluid. Hence, in embodiments, the light source may have a shape that set the buoyancy force to a specific value in a reference fluid.
The buoyancy force may be set to the specific value by the extension of the shape in vertical direction and/or by the distance along the vertical direction between surface portions of the light source that have a surface normal with a relevant vertical component, for example.
In other words, a shape that is configured for having a given buoyancy may be a shape that is oversized or undersized (if possible) with respect to the components enclosed in the light source and the size needed due to the fabrication and/or optical properties of the light source.
Alternatively or in addition, the light source may have a shape that sets at least one of the buoyancy force and the drag force to a specific value in a reference fluid.

The light emitting portion includes usually a light emitting unit, in particular a light emitting unit according to any embodiment disclosed with respect to the method, for example an LED.

The light source may include a casing that is air- and water-tight for the light source being suitable for delivering light energy in a facility for culturing an organism.

Depending on the facility, the light source is configured to work under pressure and the casing is water-tight at operational pressure of the facility.

The casing may be as disclosed above.

The light source may be considered as a device for delivering light energy or a set of parts if it includes the light emitting portion, an adjustment portion of a first kind and an adjustment portion of a second kind.

The device or set of parts may include a plurality of light emitting portion, devices of a first kind and devices of a second kind.

In an embodiment, the light source includes the adjustment portion and the adjustment portion includes at least one of:
The weight element or a further weight element.
In other words, the weight element of the light source may be arranged in or at the adjustment portion or a weight element that is in addition to a weight element arranged somewhere else in or at the light source, for example in or at the light emitting portion, may be arranged in the adjustment portion, in embodiments of the light source.
The magnet configured to interact with an external magnetic field or a further magnet configured to interact with an external magnetic field.
In other words, the magnet configured to interact with an external magnetic field may be arranged in or at the adjustment portion or a magnet configured to interact with an external magnetic field that is in addition to a magnet configured to interact with an external magnetic field arranged somewhere else in or at the light source, for example in or at the light emitting portion, may be arranged in the adjustment portion, in embodiments of the light source.
In an embodiment, a magnet configured for positioning the light source in at least one of the feeding unit, the extracting unit, the charging station, the cleaning unit, the assorting unit, the identification station, and the programming station is arranged in or at the light emitting portion and the magnet configured to interact with an external magnetic field is arranged in the adjustment portion.
A shape that alters the interaction between the light source and the fluid. In particular, the interaction is altered with respect to a light source having no adjustment portion or a different adjustment portion.
For example, the ability of the light source for laminar flow of a fluid over the light source is altered.

In an embodiment, the light source may include an adjustment portion of a first kind and an adjustment portion of a second kind, wherein the adjustment portion of the first kind distinguishes from the adjustment portion of the second kind in at least one of the weight element, the magnet, and the shape.

In any embodiment of the light source including the adjustment portion and the light emitting portion, the adjustment portion may be detachable from the light emitting portion.

The light emitting portion may be configured for attaching an adjustment portion (another or the same) to the light emitting portion after detaching an adjustment portion.

The adjustment portion may be configured for being reattached to a light emitting portion (another or the same) after its detachment from a light emitting portion.

In embodiments, the light source has a shape that favors laminar flow of the fluid over the light source.

The shape may be any shape as disclosed with respect to method and/or the facility.

In particular, the shape may be ovoid-shaped, drop-shaped or spherical.

The adjustment portion, if present, may have a shape that influences the flow of the fluid over the light source. In particular, the ability of the light source to favor laminar flow may depend on the adjustment portion. For example, the shape of the light source with attached adjustment portion may be more favorable than the shape of the light source without attached adjustment portion or with a different adjustment portion.

“Having a shape that favors laminar flow” means on the level of the light source that the surface forming the shape of the light source has an amount of surface area with a surface normal that is predominantly in a specific direction that is reduced with respect to typical shapes of light sources. The specific direction is the direction of the relative movement of the light source with respect to the fluid, when the light source is immersed in the fluid and if there is such a relative movement.

However, the appearance of turbulences, and hence increased fouling, does not only depend on the shape of the light source but also on the relative velocity between light source and fluid. Therefore, the fouling-issue may also be addressed by the light source having a shape that is configured for contributing to an adjusted (coordinated) motion between the light source and the fluid.

In other words, the shape can be configured for increasing the ability of the light source to carry out a motion that is adjusted to (coordinated with) a motion of the fluid. In particular, the shape can be configured for increasing the ability of the light source to be transported by the fluid.

Yet in other words, the shape can be configured for the force generated by the flow of the fluid over the light source being a relevant or even dominating component of the resulting force acting on the light source in the fluid.

For example, a surface area having a surface normal that is predominantly in direction of the flow of the fluid may be used for increasing or decreasing said force.

In embodiments including the light emitting portion and the adjustment portion, the ability of the light source to be transported by the fluid in motion may depend on the adjustment portion attached to the light emitting portion.

Due to the fact that a shape of the light source configured for contributing to an adjusted (coordinated) motion between the light source and the fluid is a further option to determine and/or adjust the motion of the light source in the fluid, this feature represents an option to determine the motion of the fluid in the light source in the fluid that is in addition to the option given so far. In other words, this option can be realized alone or in combination with any other option for determining the motion of the light source in the fluid in any embodiment of the light source disclosed.

In any embodiment of the light source, the light source may include one or more features used for maintenance of the light source, in particular one or more features used in the steps for maintenance disclosed with respect to the method and/or one or more features corresponding to related features of units and stations disclosed with respect to maintenance in the description of the facility.

In particular, the light source may include at least one of:
A charging unit.
In an embodiment, the charging unit is a charging unit for wireless charging of the rechargeable internal energy supply.
The charging unit may be a charging unit for inductive charging.
In an embodiment, the charging unit includes a conductive, in particular metallic, contact that is arranged at an outer surface of the light source, in particular of the light emitting portion.
In an embodiment, the light source includes the rechargeable internal energy supply and a magnet configured to position the light source during charging.
A controller configured to adjust the operational setting of the light source.
In particular, the controller is configured to communicate with an external (remote) device and to adjust the operational settings of the light source in dependence of commands received in the communication with the external device.
The communication may be wireless communication.
In an embodiment, the controller may be a programmable controller to control the energy transmitted to the fluid, wherein the controller can be programmed or re-programmed in a wireless manner, in particular through external connections.
The controller may be configured to communicate a failure of the light source to the external device.
Emitted wavelength range, emitted intensity, power delivery, length of operation, mode of dispensing light, e.g., continuous, pulsed etc., are examples of operational settings of the light source.
An electronic or optical identification unit configured for an automated identification of the light source and/or of its adjustment portion, if present. In particular, the identification unit may be configured to read-out at least one property of the light source.
The identification unit may include an RFID-tag, a bar code or a QR code, for example.
The identification unit may be configured to identify the light source when entering any units or stations of the facility arranged outside of the volume, in particular any unit or station disclosed with respect to maintenance.
The identification unit may be used to perform controlled charging and/or re-programming of the light source.
The identification unit may be an identification chip.
The magnet configured for positioning the light source. In particular, the magnet is configured for positioning the light source at least in one of the charging station, the programming station and in the cleaning unit.
The communication unit.

### BRIEF DESCRIPTION OF THE DRAWINGS

The subject matter of the invention will be explained in more detail in the following text with reference to exemplary embodiments which are illustrated in the attached drawings, which schematically show:

### BRIEF DESCRIPTION OF THE DRAWINGS

FIG. 1 is a cross-sectional view of an exemplary light source according to an embodiment of the invention;

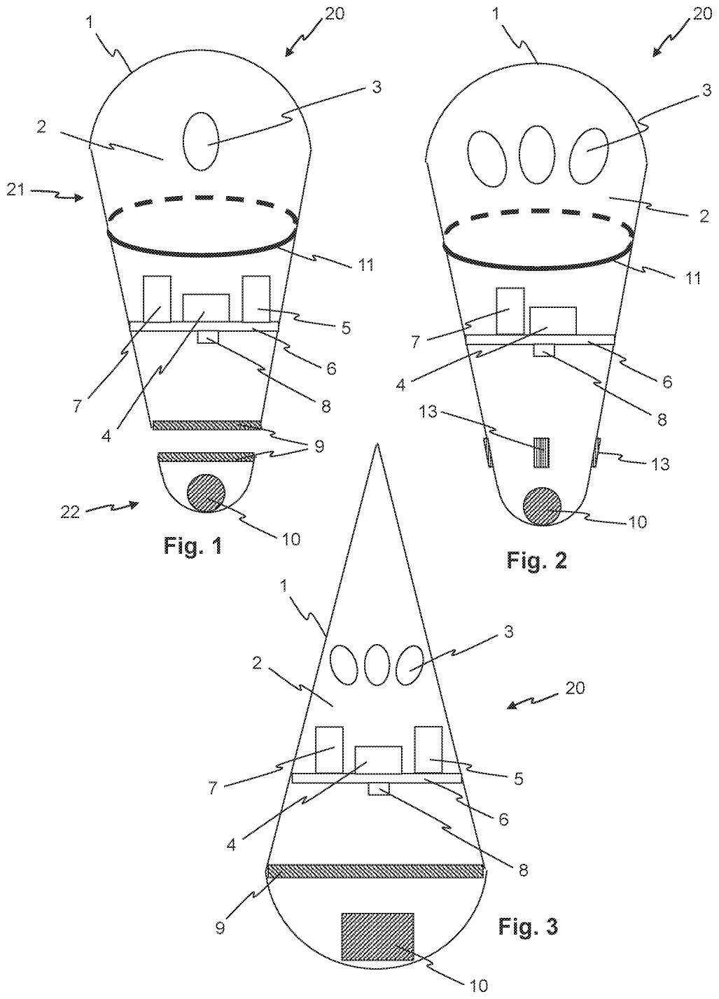

FIG. 2 is a cross-sectional view of a further exemplary light source according to an embodiment of the invention;

FIG. 3 is cross-sectional view of yet a further exemplary light source according to an embodiment of the invention;

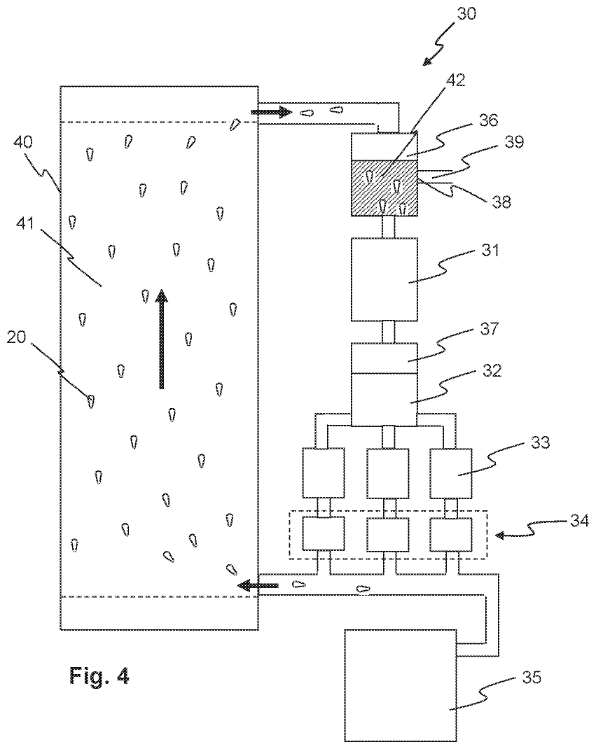

FIG. 4 is a cross-sectional view of an exemplary facility according to an embodiment of the invention;

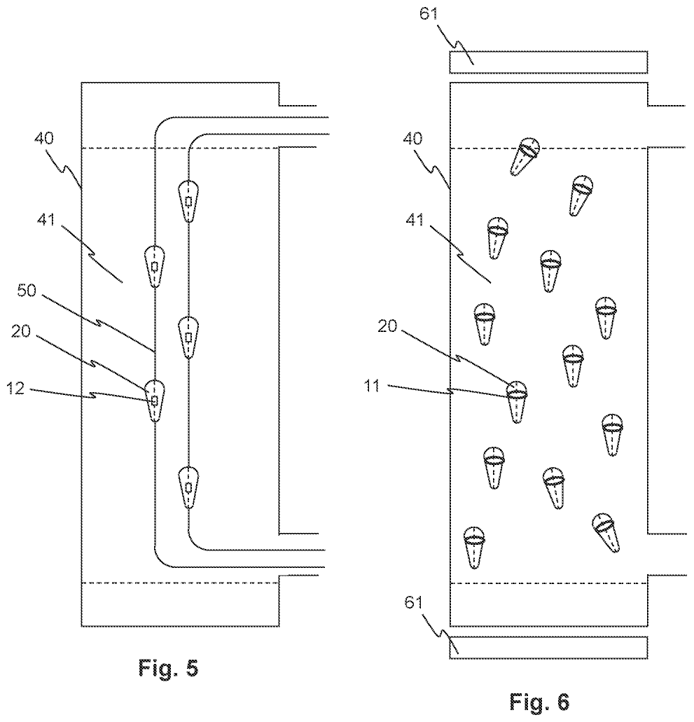

FIG. 5 is a cross-sectional view of another exemplary facility according to an embodiment of the invention; and

FIG. 6 is a cross-sectional view of yet another exemplary facility according to an embodiment of the invention.

### DETAILED DESCRIPTION OF THE INVENTION

Referring to FIG. 1, the exemplary embodiment of the light source 20 shown includes a light emitting portion 21 and an adjusting portion 22.

The light emitting portion 21 includes a casing 1 defining an interior chamber 2 including a light dispensing unit 3, a rechargeable internal energy supply 4, a charging unit 5, a microchip (controller) 6 with a communication unit 7, an identification unit 8, and a magnet 11.

The adjusting portion 22 includes a weight element 10.

The light source 20 includes further a connecting device 9 for attaching the adjusting portion 22 to the light emitting portion 21 in a releasable manner.

In the embodiment shown, the magnet 11 is a positioning magnet, this means a magnet configured to position the light source in a component of the facility in which it is used, in particular in a charging station 33.

In the exemplary embodiment shown, the light dispersing unit 3 is an LED, the rechargeable internal energy supply 4 is a battery, the identification unit 8 includes an identification chip, and the connecting device 9 includes connecting magnets or a screwing mechanism.

In the embodiment shown, the charging unit 5 is a wireless charging unit 5 that may use any optical or electromagnetic charging technology such as those used in pacemakers, mobile phones, house appliances such as toothbrushes etc, or any other wireless charging technology found more suitable for the purpose of charging the battery unit. Different to the embodiment shown, charging may be carried out by bringing a conductive contact 13 of the light source 20 in direct contact with a conductive contact of a charging station 33.

The microchip 6 is configured to provide the photosynthesizing biomass in suspension in the culture medium with a controlled lighting in terms of intensity measured in Watts, lighting time and lighting cycles (on and off).

FIG. 2 is a cross-sectional view of a further exemplary embodiment of the light source 20.

The embodiment of FIG. 2 differs from the embodiment of FIG. 1 in:
The light source 20 does not include a light emitting portion 21 and an adjustment portion 22 attached or attachable to the light emitting portion 21. Rather, the light source 20 is an integral light source 20 that forms one piece. Consequently, the light source 20 does not include any connection device for attaching the adjusting portion 22 to the light emitting portion 21.
The light source 20 includes more than one dispensing unit 3. The dispensing units 3 may differ in the wavelength range they emit, for example.
The dispensing units 3 are controllable individually via microchip (controller) 6. Optionally, the magnet 11 of the device may be strong enough for influencing the motion of the light source 20 in a fluid by an external magnetic field applied in the fluid. Due to the shown design and arrangement of the magnet 11 in the light source 20, the light source 20 has a set orientation in the external magnetic field, in particular in an external magnetic field that is essentially homogeneous.
Optional conductive contacts 13 for charging the rechargeable internal energy supply 4 by bringing at least one of the conductive contacts 12 in contact with a conductive contact of a charging station 33.

It goes without saying that the arrangement of features that determine the behaviour of the light source 20 in a fluid and the orientation of the light source 20 in a volume 41 of a facility 30, wherein an organism is influenced (for example, cultured) in the volume 41 by the electromagnetic energy emitted by the light source 20 may depend on the operation mode of the facility in which the light source 20 is used.

FIG. 3 shows a cross-sectional view of yet a further exemplary embodiment of the light source 20. In this embodiment too, the light source 20 has an exemplary shape that is ovoid-like. However, the weight element 10 is arranged opposite to the tapered end of the ovoid-like shape.

FIG. 4 shows a cross-sectional view of an exemplary facility 30 according to an embodiment of the invention.

The facility 30 includes an outer wall 40 surrounding a volume 41 including the culture milieu, this means a liquid including the organism to be cultured.

In the embodiment shown, light sources 20, for example light sources the one disclosed in FIG. 1 or 2, are swimming freely in the liquid.

The facility shown is a closed-type photobioreactor (PBR) including components for the maintenance of the light sources. Further, the facility shown is a closed system.

The components for the maintenance of the light sources are:
A light source extracting unit 36.
In the embodiment shown, the light source extracting unit 36 includes a settling chamber from which harvested micro algae culture 42 can be extracted via an extracting pipe 39 while the light sources 20 cannot enter the extracting pipe 39, for example due to a filter 38 arranged at the inlet to the extraction pipe.
The light source extracting unit 36 can be a decanter.
A cleaning unit 36 in which the light sources 20 are cleaned from surface adhesions, for example by ultrasonic cleaning.
An identification station 37 in which the kind of the light emitting portion 21 and/or the adjustment portion 22 of a light source 20 is identified, for example by an RFID or chip of the light emitting portion 21 or adjustment portion 22.
A sorting unit 32 in which the different kinds of light sources identified in the identification station 37 are separated.
Charging stations 33 in which the batteries 4 of the light sources are reloaded. Optionally, the operational parameters of the light source 20 can be adapted, for example adapted to current medium conditions via the controller 6, for example by use of a feedback-loop. However, adaption of the operational parameters may be carried out elsewhere in the facility 30.
In particular, the charging station 33 may include a programming station. However, the programming station may be arranged elsewhere in the facility and/or be a separate station, in particular a separate station of components for the maintenance of the light sources.
A light source feeding unit 34 that feeds back the light sources 20 to the volume 40 in which culturing of the organism takes place.
The light source feeding unit 34 can be configured, for example programmed, to feed light sources 20 in proportion of the different wavelength ranges needed in the volume 40 for culturing.
In other words, the sorted light sources 20 may differ in the wavelength range they emit and the light source feeding unit 34 may be configured to feed amounts of the different light sources 20 into the volume 40 that correspond to the mixture of wavelengths ranges needed.

In a method related to the facility 30 shown in FIG. 4, the light sources 20 travel to the components in the above-listed sequence. The facility 30 includes other components usually present in facilities for culturing an organism, such as a feed preparation unit 35 for feeding the volume 40 with nutriments.

The arrows shown in FIG. 4 indicate the manner the light sources and the liquid travels in the facility 30 shown.

In a variant of the embodiment shown in FIG. 4, the light source 20 includes a light emitting unit 3 that is configured to emit a first wavelength range and a second wavelength range and to be operated at the first wavelength range or the second wavelength range. Alternatively, the light source 20 may include a plurality of light emitting units 3 that emit at different wavelength ranges.

In this variant, the facility 30 does not need the identification station 37, the sorting unit 32, and a light source feeding unit 34 that is configured to feed into the volume 41 a set number of light sources that have been sorted in dependence of the light they emit. Instead, the facility 30 includes a “reprogramming unit” in which the operational parameters, in particular the emitted wavelength range, are adjusted, if needed. In particular, the microchip 6 may be “reprogrammed” via its communication unit 7.

FIG. 5 shows a cross-sectional view of another exemplary facility 30 according to an embodiment of the invention. The part of the facility 30 in which the organism is cultures is shown only.

The embodiment shown differs from the embodiment according to FIG. 4 in particular in that the light sources 20 are not swimming freely in the volume 41, but that they are engaged to a guiding element 50 via an engagement portion 12 of the light source 20.

The engagement of the light source 20 is such that the light source 20 can move along the guiding element 20, only. This movement can be a movement resulting from the light element 20 swimming freely in the direction along the guiding element 50 or it can be a movement resulting from a movement of the guiding element 50. The latter means that the light source 20 is pulled through the volume 41.

In the embodiment shown, the guiding element 50 is a guiding wire.

The guiding element 50 is formed according to the flow field of the liquid through the volume 41.

FIG. 6 shows a cross-sectional view of yet another exemplary facility 30 according to an embodiment of the invention. The part of the facility 30 in which the organism is cultures is shown only.

The embodiment shown differs from the embodiment according to FIG. 4 in particular in that the facility 30 includes an element 61 for generating a magnetic field in the volume 41. In addition, the magnet 11 of the light source 20 is a magnet adjusted to the field strength generated by the element 61 for generating the magnetic field in the volume 41 such that the movement of the light source 20 in the fluid may be influenced.

1. A light source for delivering electromagnetic energy in a facility for influencing an organism with electromagnetic energy, the light source comprising a rechargeable internal energy supply or the light source does not comprise a rechargeable internal energy supply and being configured to establish a wired connection to an external energy supply, wherein a motion of the light source in a fluid is determined by the light source comprising at least one of:
a light emitting portion and an adjustment portion attachable to the light emitting portion;
an engagement portion configured to engage the light source with a guiding element;
a magnet configured to interact with an external magnetic field; and
a weight element.

2. The light source according to claim 1, comprising the adjustment portion, wherein the adjustment portion comprises at least one of the weight element or a further weight element, the magnet or a further magnet, and a shape that alters the interaction between the light source and the fluid.

3. The light source according to claim 1, comprising the adjustment portion and the light emitting portion, wherein the adjustment portion is detachable from the light emitting portion.

4. The light source according to claim 1, wherein the light source has a shape that favors laminar flow of the fluid over the light source.

5. The light source according to claim 1, wherein the light source comprises at least one of:
an identification unit for an automated identification of the light source,
a programmable controller to control the energy transmitted to the fluid, wherein the controller can be programmed or re-programmed in a wireless manner,
the rechargeable internal energy supply and a magnet configured to position the light source during at least one of charging, programming or reprogramming, and cleaning.

6. A device for delivering electromagnetic energy in a facility for influencing an organism with electromagnetic energy, the device comprising a light source, wherein the light source comprises a rechargeable internal energy supply or the device comprises a wired connection of the light source to a power supply that is outside of the light source, wherein at least one of the following applies:
the light source comprises a light emitting portion and an adjustment portion for adjusting a motion of the light source in a fluid, wherein the adjustment portion is attached or attachable to the light emitting portion;
the device comprises a plurality of light sources that differ in their motion in a fluid;
the light source comprises an engagement portion configured to engage the light source with a guiding element of the device, wherein the guiding element defines a direction of motion along which the engaged light source can move and the engagement portion is configured to move the engaged light source together with the guiding element along the direction or to allow a movement of the engaged light source along the guiding element in the direction;
the light source comprises a magnet configured to interact with an external magnetic field in a manner that a motion of the light source in a fluid can be influenced;
the light source comprises a weight element.

7. A facility for influencing an organism with electromagnetic energy, the facility comprising an outer wall, a volume for influencing the organism with the electromagnetic energy, a fluid arranged in said volume, wherein the fluid comprises the organism, and a device for delivering electromagnetic energy to the fluid, wherein the device for delivering electromagnetic energy comprises a light source that is arranged in the fluid, wherein the light source comprises a rechargeable internal energy supply or a wired connection to a power supply that is outside of the light source, wherein the light source can move in the fluid, wherein a motion of the light source in at least one direction in the fluid is determined by at least one of:
the light source being a light source adjusted to a characteristic of the fluid;
the light source being a light source selected from a plurality of light sources in dependence of a characteristic of the fluid;
the facility comprising a guiding element, wherein the light source and the guiding element are configured for applying a pulling force to the light source via the guiding element;
a magnetic field applied in the volume, wherein the light source comprises a magnet.

8. The facility according to claim 7, wherein the motion of the light source is determined by a resulting force acting in the fluid on the light source.

9. The facility according to claim 7, wherein the determined motion comprises a determined velocity differential between the light source and the fluid.

10. The facility according to claim 7, wherein the motion of the light source is determined by the light source being adjusted and/or selected for at least one of a given weight force and a given buoyancy force acting on the light source in the fluid.

11. The facility according to claim 7, wherein the light source comprises a light emitting portion and an adjustment portion, wherein the motion of the light source in the at least one direction in the fluid is determined by the adjustment portion.

12. The facility according to claim 7, wherein the facility comprises at least one of a light source feeding unit configured to feed the light source into the volume in an automated manner and a light source extracting unit configured to extract the light source from the volume in an automated manner.

13. The facility according to claim 7, wherein the facility comprises at least one of:
a charging station arranged outside of the volume;
a cleaning unit arranged outside of the volume;
an identification station configured to determine at least one property of the light source, wherein the light source comprises an electronic or optical identification unit and the identification station is configured to determine the at least one property by reading-out the identification unit and/or wherein the identification station is configured to determine a wavelength emitted by the light source;
a sorting unit configured to separate a light source of a first kind from a light source of a second kind;
a programming station.

14. A method of operating a facility for influencing an organism with electromagnetic energy, the facility comprising an outer wall and a volume for influencing the organism with the electromagnetic energy, wherein the method comprises a step of providing a fluid in the volume, wherein the fluid comprises the organism, a step of providing a light source in the fluid for delivering electromagnetic energy to the fluid, wherein the light source comprises a rechargeable internal energy supply or a wired connection to a power supply that is outside of the light source, wherein the light source is provided in the fluid in a manner that it can move in the fluid, wherein the method comprises a step of determining a motion of the light source in at least one direction in the fluid by at least one of:
a step of adjusting the light source;
a step of selecting a light source from a plurality of light sources that differ in their motion in the fluid;
a step of providing a guiding element and applying a pulling force to the light source via the guiding element;
a step of applying a magnetic field in the volume, wherein the light source comprises a magnet.

15. The method according to claim 14, wherein the method comprises at least one of the step of adjusting the light source, wherein the step of adjusting the light source is a step of adjusting the light source to a characteristic of the fluid, the step of selecting a light source, wherein the step of selecting the light source comprises a selection in dependence of a characteristic of the fluid, the step of applying a pulling force to the light source, wherein the pulling force applied depends on a characteristic of the fluid, and the step of applying a magnetic field, wherein a force generated on the light source by the magnetic field applied depends on a characteristic of the fluid.

16. The method according to claim 14, wherein a resulting force acting on the light source in the fluid is determined in the step of determining the motion of the light source.

17. The method according to claim 16, wherein the resulting force is determined to set a velocity differential between the light source and the fluid.

18. The method according to claim 14, wherein at least one of the weight force and the buoyancy force acting on the light source in the fluid is determined in the step of determining the motion of the light source.

19. The method according to claim 14, comprising at least one of the step of adjusting the light source, wherein the light source is adjusted by attaching an adjustment portion to the light source or by replacing an adjustment portion of the light source, and the step of selecting a light source, wherein the selected light source differs from the non-selected light sources of the plurality of light sources in an adjustment portion.

20. The method according to claim 14, comprising at least one of a step of feeding a light source into the volume in an automated manner and a step of extracting a light source out of the volume in an automated manner.

21. The method according to claim 14, comprising a step of maintenance of the light source provided in the fluid during operation of the facility, wherein the step of maintenance comprises at least one of:
a step of charging the light source, wherein the light source is charged outside of the volume;
a step of cleaning the light source, wherein the light source is cleaned outside of the volume;
a step of providing a plurality of separate light sources of a first kind and a plurality of separate light sources of a second kind and a step of assorting the light sources of the first and second kinds;
a step of identifying the light source;
a step of adjusting an operational setting of the light source.
